(19) United States
(12) Patent Application Publication
(10) Pub. No.: US 2005/0070471 A1
(43) Pub. Date: Mar. 31, 2005

(54) Use of coagulant-active antithrombin iii for the therapy of angiogenesis-dependent diseases

(76) Inventors: Ilhan Celik, Ebsdorfergrund (DE); Oliver Kisker, Rossdorf (DE)

Correspondence Address: Clark & Brody, 1750 K Street, N.W., Suite 600, Washington, DC, 20006, US

(21) Appl. No.: 10/497,724

(22) PCT Filed: Dec. 4, 2002
(86) PCT No.: PCT/DE02/04438

(30) Foreign Application Priority Data
Dec. 7, 2001 (DE) 101 60 133.6

(51) Int. Cl. A61K 38/46

(52) U.S. Cl. 514/12; 435/69.1

(57) ABSTRACT

The present invention relates to a method for the production of antithrombin III with antiangiogenic activity from coagulation-active (native) antithrombin III and the utilisation of coagulation-active antithrombin III for the prophylaxis and therapy of diseases.

The present invention relates to a method for the production of antithrombin III with antiangiogenic activity from coagulation-active (native) antithrombin III and the utilisation of coagulation-active antithrombin III for the prophylaxis and therapy of diseases.

### CURRENT TECHNICAL STATE OF THE ART

It is generally accepted that tumour growth, chronic inflammation, and retinopathy are angiogenesis-dependent. Herein, the balance between positive and negative regulators of angiogenesis plays a significant part. For example, tumours express positive regulators but can additionally be involved in the generation of negative regulators of angiogenesis as well. Herein, tumours secrete enzymes which proteolytically cleave such known proteins such as collagen XVIII and antithrombin III (AT III) and alter their conformation (e.g. into latent AT III) respectively. The fragments (cleavage products) thus derived then feature potent antiangiogenic properties in vitro and in vivo. Examples include angiostatin as a cleavage product from plasminogen, endostatin as a cleavage product form collagen XVIII, antiangiogenic antithrombin III as a cleavage product from antithrombin III, and conformationally changed antithrombin III (O'Reilly et al. Science 1999; Kisker et al. Cancer Research 2001).

Furthermore, it is known that by chemical (ex vivo) modification (citrate treatment) of native AT III, latent AT III is developed which has antiendothelial (in vitro) and antiangiogenic (in vivo) properties (O'Reilly et al. Science 1999; Larsson et al. Cancer Research 2000 and Larsson et al JBC 2001).

The list of references provides an overview of the technical state of the art:
Kisker et al. Cancer research 60, 2001, 7298-7304
O'Reilly et al. Science Vol. 285, 1999 1926-1928
Larsson et al. Cancer Research 2000, 6723-6729
Larsson et al. Journal of Biological Chemistry Vol. 276, No.15 2001, 11996-12002

The cleavage products with antiendothelial or antiangiogenic properties can be administered to the patient in the form of medicinal products. However, the production of these antiangiogenic cleavage products can currently only occur by means of technologically highly elaborate, expensive production methods, e.g. genetic modification (e.g. endostatin); besides, they are suitable to be applied to patients only under certain conditions since—depending on the expression system utilised—the proteins produced by genetic modification do not show exact congruence and 100% effectiveness in comparison to naturally occurring cleavage products.

In addition, the availability of these substances is limited by the additional steps in the procedure, and for the production of medicinal products, every new technological step in the procedure means an increase of the product's price.

The objective of the invention was to develop a method in which a substance, which does not dispose of any antiangiogenic characteristics in itself, is transformed into a cleavage product with antiangiogenic characteristics which can be utilised in the production of a medicinal product for the prophylaxis and therapy of angiogenesis-dependent diseases. Moreover, its objective was to identify a substance of this type which is suitable for the production of a medicinal product for the prophylaxis and therapy of diseases, particularly angiogenesis-dependent diseases, and to develop a test method suitable for the in-vitro identification and production of new negative and positive regulators of angiogenesis or, alternatively, for the test of different tumour cell lines.

The objective is achieved, based on the current invention, by claims 1 to 9.

Tumours and/or tumour cells secrete enzymes (e.g. proteolytic enzymes such as matrix metalloproteinases) which are able to cleave off substances with antiangiogenic activity from known proteins or to alter the protein in its protein structure in such a way that the derived altered protein has potent antiangiogenic properties.

Surprisingly, it has been found that antiangiogenic antithrombin III is a cleavage product of this kind, i.e. a conformationally altered protein from native antithrombin III. In vivo—when the tumour cells are able to cleave antithrombin III or modified its configuration—the application of native antithrombin III leads to a reduction and/or a standstill in growth due to an inhibition of vascular growth because the tumour produces large quantities of antiangiogenic antithrombin III for itself from the surplus supply of antithrombin III; thereby, the balance between positive and negative regulators is shifted in favour of the negative regulators. This means that a protein such as native antithrombin III which is available in satisfactory and/or sufficient quantities can be utilised in therapy immediately without extensive laboratory technological methods, additional steps in the procedure, and costs. Besides, separate authorisation as a medicinal product, which would be expensive, elaborate, and time-consuming, is not necessary since native antithrombin III, for instance, is a medicinal product which is already available on the market. DE19937656A1 and EP1075840A3 suggest the utilisation of antithrombin for the prophylaxis and therapy of diseases. In these, however, the possible field of application is restricted to inflammatory reactions and the function of antithrombin III is connected to leukocyte migration.

The present invention shows that the human pancreatic carcinoma cell line BxPC-3 is able to produce both cleaved and antithrombin III (latent AT III) with antiangiogenic activity modified in configuration. In a murine experiment, the therapy of a BxPC-3 and ASPC-1 tumour of 100 mm³ with 50 mg/kg/day of cleaved human antithrombin III, respectively, leads to a distinct inhibition of angiogenesis (reduced microvascular density) and thereby to a complete blocking of tumour growth. Likewise, the application of 60 mg/kg/day of normal (native) AT III can lead to a distinct inhibition of angiogenesis and tumour growth (FIG. 1), while the therapy of a tumour which does not have the ability to alter native AT III is not inhibited in its growth by normal AT III either (FIG. 2).

A method based on the invention is suggested with which AT III with antiangiogenic activity can be generated from native antithrombin III in vitro through tumor cell lines. Additionally, this method can be utilised for the in vitro identification and production of new negative and positive regulators of angiogenesis or, alternatively, for testing different tumour cell lines.

Moreover, the utilisation, based on the invention, of coagulation-active AT III as a medicinal product for the prophylaxis and therapy of oncological diseases, particularly solid tumours and leukemias, is shown.

The plasma protein antithrombin can exist in different conformations and variations.

Based on the present invention, coagulation-active AT III refers to the native antithrombin III existent in the body which is active during blood coagulation. Antithrombin III with antiangiogenic activity, however, refers to the conformationally altered native antithrombin III, the latent and cleaved antithrombin III, neither of which have a function during blood coagulation.

The invention relates to the administration of coagulation-active antithrombin III to patients as a medicinal product, wherein the term “patient” pertains to humans and vertebrates alike. Hence, the medicinal products can be utilised in human and veterinary medicine.

The antithrombin III with antiangiogenic and therapeutic activity of the present invention is administered to the patients, as part of a pharmaceutically acceptable composition, either orally, rectally, parenterally, intravenously, intramuscularly, or subcutaneously, intracisternally, intravaginally, intraperitoneally, intravascularly, intrathecally, intravesically (by instillation into the urinary bladder), locally (powder, ointments, or drops), or in the form of a spray (aerosol). The intravenous, subcutaneous, intraperitoneal, and intrathecal administration can be carried out continuously by means of a pumping or dosing unit.

Pharmaceutically acceptable composition can include modifications as salts, esters, amides, and prodrugs, as long as they do not, according to reliable medical assessment, cause excessive toxicity, irritations, or allergic reactions in the patient.

Forms of dosage for the local administration of the compound, included in the present invention, include ointments, powder, sprays, or inhalants.

The active compound is mixed, under sterile conditions, with a physiologically active carrier and possible preservatives, buffers, or propellants, according to requirements.

### PRACTICAL EMBODIMENTS

A) Generation of AT III with Antiangiogenic Activity, Cleaved and Latent AT III in vitro by Tumour Cell Lines

Methods: Human pancreatic carcinoma cell lines BxPC3 and ASPC-1 are cultivated in a cell culture (medium: RPMI 1640+10% FCS+1% Penicillin/Streptomycin; 37° C. and 5% CO₂). In order to verify whether the serum-free cell medium of these cell lines has the ability to modify the configuration of human native AT III, the cells are grown to confluence. Subsequently, the cells are rinsed with PBS twice and incubated with a serum-free medium (RMPT 1640) for 48 hours (so-called conditioned medium). Afterwards, 1 mg/ml of native human AT III is added to the serum-free medium. After a further incubation period of 48 hours, the medium supernatant is extracted and subsequently applied to a 72 hour lasting bFGF-stimulated endothelial cell proliferation assay, which is known to the person skilled to the art. In addition, the conformation alteration of the AT III in comparison to human native AT III is examined by Western Blot Analysis with an antibody against human native AT III and by urea-gradient gel analysis.

Result: The supernatant of the BxPC-3 cells shows a concentration-dependent inhibition of the proliferation rate in the endothelial cell proliferation assay. The serum-free medium without added native AT III (control) does not exhibit any inhibitory properties. Likewise, the addition of AT III to a pure medium (without preincubation of the cells=negative control) does not show any antiproliferative properties. In the Western Blot Analysis of the conditioned medium with native AT III the cleaved form of AT III is detectable. Additionally, a latent form of AT III is detectable in urea-gradient gel analysis. After purification with a heparin-sepharose column (50 mM Tris buffer pH 7.4), the AT III with antiangiogenic activity elutes at 0.5 molar NaCl. The further purification occurs via anion exchange column (Mono-Q column) with 20 mM Tris buffer, pH 7.0, and elution by means of a gradient from 0 to 0.35 molar NaCl. Here, AT III with antiangiogenic activity elutes at 0.31-0.35 molar NaCl, latent and cleaved AT in separate fractions. The AT III with antiangiogenic activity shows antiendothelial effectiveness in the endothelial cell proliferation assay.

In the identical experimental approach (cf. above), no inhibitory activity of the conditioned medium is found for the cell line ASPC-1 after adding native AT III in the endothelial cell proliferation assay. All of the positive and negative controls do not display any inhibitory activity either. Western Blot Analysis and urea-gradient gel analysis detect neither a cleaved nor latent AT III for this cell line. Hence, the cell line ASPC-1 does not dispose of the necessary enzymatic activity of generating AT III with antiangiogenic activity from coagulation-active AT III. With the present method, a number of tumour cell lines can be tested for their enzymatic activity of generating AT III with antiangiogenic activity from coagulation-active AT III. It is also possible to test further protein candidates for their ability to be altered into positive or negative regulators of angiogenesis by a given tumour cell line.

The method presented shows that, following the addition of proteins without antiangiogenic properties to the conditioned medium (cf. above) of tumour cells, the latter are altered in their configuration and/or structure in such a way that positive or negative regulators of angiogenesis are derived. The new substances which have thus been generated can be identified in subsequent steps of purification (e.g. gel analysis) and utilised, after adequate purification, for the utilisation in vivo as a medicinal product in the prophylaxis and therapy of diseases.

B) Coagulation-Active AT III as a Medicinal Product in the Therapy of Two Human Pancreatic Carcinoma Cell Lines (BxPC3 and ASPC-1) in the Tumour Mouse Model (SCID Mouse)

Method: The two (BxPC3 and ASPC-1) human pancreatic carcinoma cell lines are cultivated in the cell culture (cf. Section A). The cells are harvested and 5×106 cells/mouse are implanted sc on the backs of the SCID mice. After the tumours have reached a size of c. 100 mm³, the mice are randomised in 3 groups per cell line (n=4 or 7/group).

Afterwards, the sc injection of 60 mg/kg body weight of latent (AT III with antiangiogenic activity) and/or native AT III (coagulation-active AT III) is administered far away from the tumour. As a control, sodium chloride (NaCl 0.9%) or BSA (Bovines Serum Albumin 60 mg/kg body weight) is injected. The injections are given daily at the dosages listed. The tumour volume is determined every 3-5 days and stated in relation to the tumour volume of the control group at the end of the experiment (BxPC3 after 26 days and ASPC-1 after 11 days). Therein, a quotient is formed by dividing the tumour volume of the control group (T/C) from the tumour volume of the therapy group

Results: The human pancreatic carcinoma cell line BxPC3 can be inhibited in its tumour growth by adding latent (with antiangiogenic activity) and native AT III (coagulation-active). For latent AT III, this results in a T/C of 0.1 (90% inhibition in comparison to the control group). For native AT III, it results in a T/C of 0.15 (85% inhibition in comparison to the control group) (cf. FIG. 1).

For the human pancreatic carcinoma cell line ASPC-1, a T/C of 0.31 (69% inhibition of tumour growth in comparison to the control group) is found in the experimental approach described above. For native AT III, no significant inhibition of tumour growth can be detected (T/C=0.94 or 6% inhibition of the tumour volume in comparison to the control group) (cf. FIG. 2).

### FIGURES

FIG. 1 Therapy of the human pancreatic carcinoma cell line BxPC3 in the tumour mouse model (SCID mouse) with native and latent AT III (60 mg/kg body weight) versus control (60 mg/kg body weight BSA=Bovine Serum Albumin). Production of T/C (with antiangiogenic activity)=quotient from the tumour volume of the therapy group, divided by the tumour volume of the control group (BSA). Antithrombin III from the human pancreatic carcinoma cell line BxPC-3

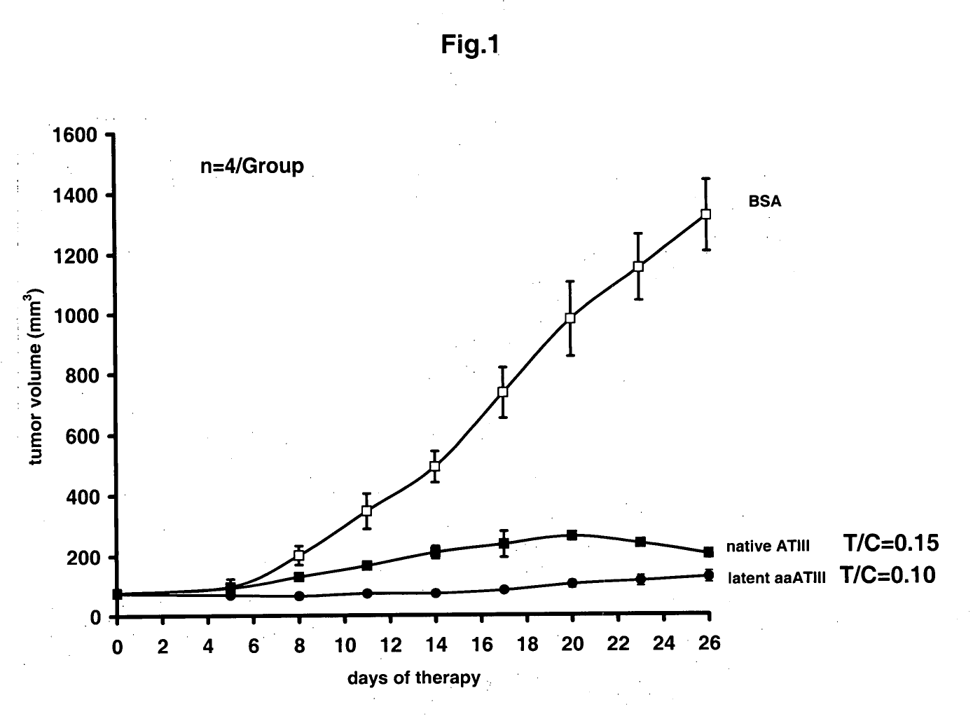

FIG. 2 Inhibition of the angiogenesis with human antithrombin III with antiangiogenic activity, therapy of the human pancreatic carcinoma cell line ASPC-1 in the tumour mouse model (SCID mouse) with native and latent AT III (60 mg/kg body weight) versus control (NaCl 0.9%). T/C=quotient from tumour volume of the therapy group, divided by tumour volume of the control group (NaCl).

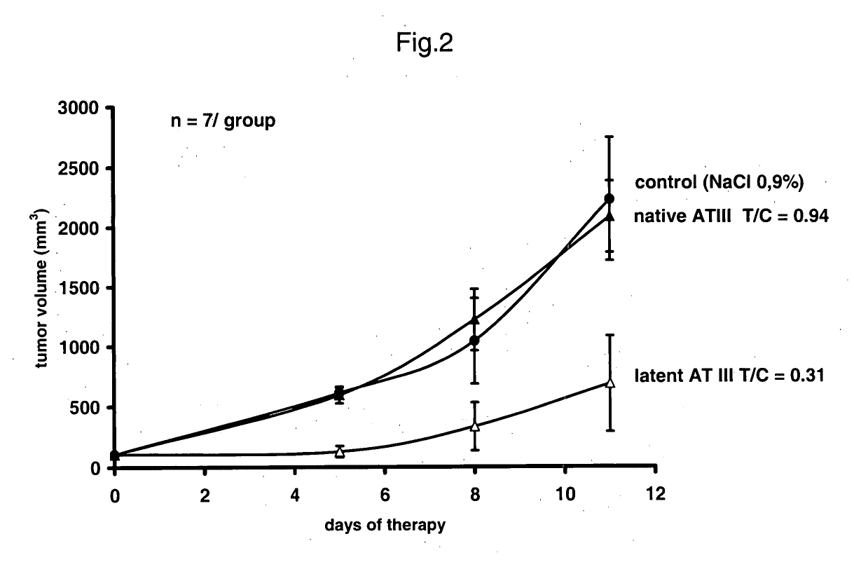

1. Method for the production of AT III with antiangiogenic activity, characterised by the following steps:
cultivation of the tumour cell line up to confluence
incubation with a serum-free medium for 48 hours
addition of 1 mg/ml of native human AT III
incubation for 48 hours
extraction of the medium supernatant
realisation of the bFGF-stimulated endothelial cell
proliferation assay
analysis of the AT III with antiangiogenic activity by Western Blot Analysis and urea-gradient gel analysis purification with heparin-sepharose column and anion exchange column

2. Utilisation of the method according to claim 1 for the in-vitro identification and production of new negative and positive regulators of angiogenesis.

3. Utilisation of the method according to claim 1 for the in-vitro identification of different tumour cell lines for their ability of altering proteins' conformation in such a way that they are effective as negative or positive regulators of the angiogenesis.

4. Utilisation of coagulation-active antothrombin III for the production of a medicinal product for the prophylaxis and therapy of diseases, particularly angiogenesis-dependent diseases.

5. Utilisation according to claim 4, characterised by the fact that oncological diseases are treated.

6. Utilisation according to claims 4 to 5, characterised by the fact that diseases involving solid tumours and leukaemia are treated.

7. Utilisation according to claims 4 to 6, characterised by the fact that coagulation-active antithrombin III is utilized intravenously, subcutaneously, intraperioneally, intrathecally, intravesically (by instillation into the urinary bladder), and topically.

8. Utilisation according to claims 4 to 7, characterised by the fact that coagulation-active antithrombin III is utilised as aerosol.

9. Antithrombin III with antiangiogenic activity, characterised by methods according to claim 1.
